(19) United States
(12) Patent Application Publication
(10) Pub. No.: US 2022/0307077 A1
(43) Pub. Date: Sep. 29, 2022

(54) CONSERVATIVE CONCURRENT EVALUATION OF DNA MODIFICATIONS

(71) Applicant: AMBRY GENETICS CORPORATION, Aliso Viejo, CA (US)

(72) Inventors: Brady CULVER, Aliso Viejo, CA (US); Daniella BIANCHI-FRIAS, Aliso Viejo, CA (US)

(21) Appl. No.: 17/702,530

(22) Filed: Mar. 23, 2022

Related U.S. Application Data
(60) Provisional application No. 63/165,332, filed on Mar. 24, 2021.

(51) Int. Cl. C12Q 1/6827; C12Q 1/6869; C12Q 1/6806

(52) U.S. Cl. CPC C12Q 1/6827; C12Q 1/6869; C12Q 1/6806; C12Q 2523/125; C12Q 2600/154

(57) ABSTRACT

Disclosed herein are methods for performing a medical procedure by determining the mutation and DNA modification status of a biological sample from a subject with respect to a single sample. Also disclosed herein are methods for diagnosis by determining whether a DNA of a biological sample contains nucleobase modifications and mutations, by assessing the DNA modification and mutation status with respect to a single sample.

### CROSS-REFERENCE TO RELATED APPLICATION

This application claims priority from U.S. provisional patent application No. 63/165,332 filed on Mar. 24, 2021. The entire contents of this earlier filed application are hereby incorporated by reference in their entirety.

### FIELD

The methods relate to determining whether a DNA has been modified by evaluating DNA modification status with respect to a single sample.

### BACKGROUND

In biology, epigenetics is the study of heritable phenotype changes that do not involve changes in DNA sequences. The epigenetic changes involve changes that affect gene activity and expression.

Epigenetics also refers to the changes that may be functionally relevant to the genome and involve chemical modifications of nucleic acids. Examples of the chemical modifications include methylation, hydroxy methylation, and strand specific DNA deamination. These chemical modifications do not change the gene sequence, but are known to affect gene expression. These epigenetic changes may last through for the duration of the cell's life, or even through the cell lineage.

Cell free nucleic acids, including cell-free DNA (cfDNA), have become increasingly important in the medical diagnostic industry. Using cfDNA in medical diagnostic tests offers several advantages, chiefly in that it allows obtaining and using DNA present in body fluids without requiring extensive biopsies of tissues. In recent years, cfDNA has gained prominence in the area of biomarker diagnosis for cancer, pre-natal diagnosis, and other medical conditions.

The disadvantage of cfDNA is the relatively low amount of cfDNA present within the blood sample of a patient. For instance, a typical cfDNA concentration is as low as 1 ng/ml. Alborelli I. et al. (2019). This relatively low amount impairs the ability of researchers and clinicians to collect enough material for testing or detecting the presence of DNA modifications. Therefore, the ability to conduct multiple analyses on the same sample is extremely useful.

### SUMMARY

The present disclosure provides methods for performing a medical procedure by determining the mutation and DNA modification status of a nucleic acid sample from a subject with respect to a single sample. The present disclosure also provides methods for diagnosis by determining whether a DNA of a nucleic acid sample contains nucleobase modifications and mutations, by assessing the DNA modification and mutation status with respect to a single sample. This disclosure sets forth processes, in addition to making and using the same, and other solutions to problems in the relevant field.

In some embodiments, there is provided a method for assessing DNA modification status of a nucleic acid sample from a subject with respect to a single sample, the method comprising: (a) a step of evaluating DNA modification status of a DNA fragment in a nucleic acid sample in order to assess the DNA modification status of a biological samples. In certain embodiments, the method comprises (b) a step of evaluating DNA mutation status of the DNA fragment in the nucleic acid sample before, during, or after step (a). In certain embodiments, the method comprises (b) a step of evaluating DNA mutation status of the DNA fragment in the nucleic acid sample before step (a). In certain embodiments, the method comprises (b) a step of evaluating DNA mutation status of the DNA fragment in the nucleic acid sample during step (a). In certain embodiments, the method comprises (b) a step of evaluating DNA mutation status of the DNA fragment in the nucleic acid sample after step (a). In certain embodiments, the method comprises (c) a step of producing a copied DNA fragment of the DNA fragment in the nucleic acid sample before step (a). In certain embodiments, the method comprises in step (a) the DNA modification status of the DNA fragment in the nucleic acid sample is evaluated basing on methylations, hydroxy methylations, strand specific deaminations or presence of N-methyladenine bases occurred to the DNA fragment. In certain embodiments, the method in step (b), DNA mutation status of the DNA fragment in the nucleic acid sample is evaluated basing on single nucleotide variations, insertions, translocations, copy number, or deletions present in the DNA fragment. In certain embodiments, the method in step (c), the copied DNA fragment is produced by immobilizing the DNA fragments to a substrate by: (a) immobilizing the DNA fragment to a substrate to create an immobilized DNA fragment; (b) copying the DNA fragment through PCR amplification to create a copied DNA fragment; and (c) separating the copied DNA fragment from the immobilized DNA fragment. In certain embodiments, the method comprises each strand of the DNA fragment is immobilized separately to the substrate. In certain embodiments, the method comprises a step (d) of obtaining a nucleic acid sample from the subject before step (a). In certain embodiments, the method comprises a step (e) of tagging a DNA fragment in the nucleic acid sample to create a tagged DNA fragment. In certain embodiments, step (e) is performed before the DNA fragment is copied, immobilized to a substrate, or before evaluating DNA mutation status. In certain embodiments, the method comprises a step of evaluating DNA mutation status of the copied DNA fragment.

In some embodiments, there is provided a method for performing a medical procedure by assessing DNA modification status of a nucleic acid sample from a subject with respect to a single sample, the method comprising: (a) a step of evaluating DNA modification status of a DNA fragment in a nucleic acid sample in order to assess the DNA modification status of a biological samples. In certain embodiments, the method comprises (b) a step of evaluating DNA mutation status of the DNA fragment in the nucleic acid sample before, during, or after step (a). In certain embodiments, the method comprises (b) a step of evaluating DNA mutation status of the DNA fragment in the nucleic acid sample before step (a). In certain embodiments, the method comprises (b) a step of evaluating DNA mutation status of the DNA fragment in the nucleic acid sample during step (a). In certain embodiments, the method comprises (b) a step of evaluating DNA mutation status of the DNA fragment in the nucleic acid sample after step (a). In certain embodiments, the method comprises (c) a step of producing a copied DNA fragment of the DNA fragment in the nucleic acid sample before step (a). In certain embodiments, the method comprises in step (a) the DNA modification status of the DNA fragment in the nucleic acid sample is evaluated basing on methylations, hydroxy methylations, strand specific deaminations or presence of N-methyladenine bases occurred to the DNA fragment. In certain embodiments, the method in step (b), DNA mutation status of the DNA fragment in the nucleic acid sample is evaluated basing on single nucleotide variations, insertions, translocations, copy number, or deletions present in the DNA fragment. In certain embodiments, the method in step (c), the copied DNA fragment is produced by immobilizing the DNA fragments to a substrate by: (a) immobilizing the DNA fragment to a substrate to create an immobilized DNA fragment; (b) copying the DNA fragment through PCR amplification to create a copied DNA fragment; and (c) separating the copied DNA fragment from the immobilized DNA fragment. In certain embodiments, the method comprises each strand of the DNA fragment is immobilized separately to the substrate. In certain embodiments, the method comprises a step (d) of obtaining a nucleic acid sample from the subject before step (a). In certain embodiments, the method comprises a step (e) of tagging a DNA fragment in the nucleic acid sample to create a tagged DNA fragment. In certain embodiments, step (e) is performed before the DNA fragment is copied, immobilized to a substrate, or before evaluating DNA mutation status. In certain embodiments, the method comprises a step of evaluating DNA mutation status of the copied DNA fragment.

In some embodiments, there is provided a method for assessing DNA modification status of a biological sample from a subject with respect to a single sample from a subject suspected of having a disease, the method comprising: obtaining a nucleic acid sample from the subject. In certain embodiments, the method comprises tagging a DNA fragment of the nucleic acid sample from the subject to create a tagged DNA fragment. In certain embodiments, the method comprises immobilizing the tagged DNA fragment to a substrate, wherein each strand of the tagged DNA fragment in the nucleic acid sample is bound separately to the substrate. In certain embodiments, the method comprises evaluating DNA mutation status of the tagged DNA fragment. In certain embodiments, the method comprises evaluating DNA modification status of the tagged DNA fragment. In certain embodiments, the method comprises assessing the DNA modification status of the nucleic acid sample.

In some embodiments, there is provided a method for performing a medical procedure by assessing DNA modification status of a nucleic acid sample from a subject with respect to a single sample from a subject suspected of having a disease, the method comprising: obtaining a nucleic acid sample from the subject. In certain embodiments, the method comprises tagging a DNA fragment of the nucleic acid sample from the subject to create a tagged DNA fragment. In certain embodiments, the method comprises immobilizing the tagged DNA fragment to a substrate, wherein each strand of the tagged DNA fragment in the nucleic acid sample is bound separately to the substrate. In certain embodiments, the method comprises evaluating DNA mutation status of the tagged DNA fragment. In certain embodiments, the method comprises evaluating DNA modification status of the tagged DNA fragment. In certain embodiments, the method comprises assessing the DNA modification status of the nucleic acid sample.

In some embodiments, there is provided a method for diagnosing a patient with a potential disease by evaluating DNA modification status of a nucleic acid sample from a subject with respect to a single sample from a subject suspected of having a disease, the method comprising: obtaining a nucleic acid sample from a subject. In certain embodiments, the method comprises tagging a DNA fragment of the nucleic acid sample from the subject to create a tagged DNA fragment. In certain embodiments, the method comprises immobilizing the tagged DNA fragment to a substrate, wherein each strand of the tagged DNA fragment in the nucleic acid sample is bound separately to the substrate. In certain embodiments, the method comprises evaluating DNA mutation status of the tagged DNA fragment. In certain embodiments, the method comprises evaluating DNA modification status of the tagged DNA fragment. In certain embodiments, the method comprises diagnosing a subject with a potential disease by evaluating the DNA modification status of the nucleic acid sample.

In some embodiments, there is provided a method for performing a medical procedure by diagnosing a patient with a potential disease by evaluating DNA modification status of a nucleic acid sample from a subject with respect to a single sample from a subject suspected of having a disease, the method comprising: obtaining a nucleic acid sample from a subject. In certain embodiments, the method comprises tagging a DNA fragment of the nucleic acid sample from the subject to create a tagged DNA fragment. In certain embodiments, the method comprises immobilizing the tagged DNA fragment to a substrate, wherein each strand of the tagged DNA fragment in the nucleic acid sample is bound separately to the substrate. In certain embodiments, the method comprises evaluating DNA mutation status of the tagged DNA fragment. In certain embodiments, the method comprises evaluating DNA modification status of the tagged DNA fragment. In certain embodiments, the method comprises diagnosing a subject with a potential disease by evaluating the DNA modification status of the nucleic acid sample.

In some embodiments, in any of the previous embodiments, the method further comprises copying the tagged cfDNA molecule through PCR amplification to produce an untagged copy; binding the tagged cfDNA molecule to the substrate; separating the tagged cfDNA from the untagged copy; and analyzing the mutation status of the unbound material.

### BRIEF DESCRIPTIONS OF THE DRAWINGS

Non-limiting embodiments of the present disclosure will be described by way of example with reference to the accompanying figures. For clarity purposes, not every component is labeled in every figure, nor is every component of each embodiment shown where illustration is not necessary to allow a person of ordinary skill in the art to understand the disclosure.

FIG. 1: Exemplary method of analyzing cfDNA. In 1, a pool of nucleic acids is extracted from a biological source to obtain the cfDNA. In 2, a methylated cytosine residue on the cfDNA is depicted. In 3, a first mutation, SNV or indel, present on both strands of a duplex fragment of cfDNA is depicted. In 4, a second mutation, SNV or indel, present on both strands of a duplex fragment of cfDNA is depicted. In 5, an adapter, which contains an anchoring sequence that is to be ligated to the cfDNA is depicted. The adapter can have various functional elements comprised within its sequence: methylated C residues, UMI, or primer binding sites. In 5, a Y-shaped adapter is depicted. In 6, an affinity tag present on one of the strands of the adapter is depicted. In 6, the affinity tag is at the 5′ end of the adapter and is a biotinylated nucleotide. In 7, cfDNA is ligated to the adapter depicted in 5. In 8, a pool of adapter-cfDNA ligation constructs resulting from the ligation reaction in 7 is depicted. In 9, a bead or other solid phase matrix with ability to bind to the affinity tag in 6 which in turn is bound to cfDNA molecules is depicted. Fragments from elements 2, 3, and 4 are bound to the bead and are in their native unmodified state. In 10, the polymerase, free dNTP, ions, and buffer are added to the immobilized cfDNA constructs to perform a primer extension reaction on the bead. In 11, a primer and first extension product are depicted. The primer is complementary to and hybridizes with an element within the free 3′ end of the ligated adapter. The dotted arrow represents extension from the primer towards to bead. In 12, a primer and second extension product are depicted. The primer is complementary to and hybridizes with an element within the free 3′ end of the ligated adapter. The dotted arrow represents extension from the primer towards to bead. In 13, a primer and third extension product are depicted. The primer is complementary to and hybridizes with an element within the free 3′ end of the ligated adapter. The dotted arrow represents extension from the primer towards to bead. In 14, primer extension products from on-bead primer extension reaction is depicted. These products are not bound by the affinity matrix. These products faithfully pass on the identity of elements 3 and 4, but not 2. In 15, primer extension products, the copies of the biological DNA template, are detected or read by various methodologies. This example focuses on DNA sequencing instruments. In 16, bead bound DNA, 9, is treated with an agent that alters its sequence, in this example, sodium bisulfite treatment is performed to deaminate unmethylated cytosines. In 17, the bead and its bound material, 9, after treatment with DNA altering agent, e.g. sodium bisulfite is depicted. In 18, a first product of DNA altering treatment, which retains the identity of the methylated cytosine is depicted. In 19, a second product of DNA altering treatment, which converts unmethylated cytosine to uracil is depicted. In 20, a third product of DNA altering treatment, which converts unmethylated cytosine to uracil is depicted. In 21, a primer and fourth extension product are depicted. The primer is complementary to and hybridizes with an element within the free 3′ end of the ligated adapter. The dotted arrow represents extension from the primer towards the bead. The extension product incorporates the complement of the methylated C residue, G, as it was not converted to uracil. In 22, a primer and fifth extension product is depicted. The primer is complementary to and hybridizes with an element within the free 3′ end of the ligated adapter. The dotted arrow represents an extension from the primer towards to bead. The extension product incorporates the complement of the Uracil (produced by conversion of the unmethylated C residue), A. In 22, a primer and sixth extension product are depicted. The primer is complementary to and hybridizes with an element within the free 3′ end of the ligated adapter. The dotted arrow represents an extension from the primer towards to bead. The extension product incorporates the complement of the Uracil (produced by conversion of the unmethylated C residue), A. In 24, a pool of extension products, 21, 22, and 23 are depicted. In 25, extension products from altered DNA can be detected or read by various methodologies. This depiction focuses on DNA sequencing instruments, but can be applied to any other detection method.

FIG. 2: Alternative method of analyzing cfDNA. In 27, the products of amplification of 8 (FIG. 1) are depicted. The template molecules retain original adapter, 5, and affinity tag, 6, while amplification products do not. In 28, the amplification product, 26, is mixed with affinity matrix to separate original tagged biological DNA, 8, from amplification products is depicted. In 29, unbound constructs comprising the amplification products of 8 are depicted. These constructs retain information about DNA sequence identity, but not modification status, e.g. methylation.

FIG. 3: Barplot showing measured methylation percent of a BRCA1 promoter locus in OVCAR8 or SW620 DNA following enzymatic conversion of unmethylated Cytosine to Uracil. The “_enz_beads” suffix refers to material that had been labeled with biotinylated adapter at ligation, amplified by PCR, and then captured on Streptavidin beads for the cytosine conversion step. The “enz ctrl” suffix refers to material that was not amplified or captured on beads. The low % Methylation observed for SW620 material is an indicator of the efficiency of Cytosine conversion as this material is expected to lack any methylation at BRCA1 promoter. The expectation was to see 66% methylation for OVCAR8 cell line, which is represented by an approximately 1 Ct difference between methylated and unmethylated product.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

As used herein, the term “biological sample,” refers to a sample derived from, obtained by, generated from, provided from, take from, or removed from an organism; or from fluid or tissue from the organism. Biological samples include, but are not limited to synovial fluid, whole blood, blood serum, blood plasma, urine, sputum, tissue, saliva, tears, spinal fluid, tissue section(s) obtained by biopsy, cell(s) that are placed in or adapted to tissue culture, sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, samples that can provide nucleic acids for analysis. Biological samples include, but are not limited, any derivative or fraction of the aforementioned biological samples. In certain embodiments, the term “biological sample” is synonymous with the term “nucleic acids.”

As used herein, the term “medical procedure” is also synonymous with treatment.

As used herein, the term “treatment” or “treating” of any disease or disorder refers, in certain embodiments, to ameliorating a disease or disorder that exists in a subject. In another embodiment, “treating” or “treatment” includes ameliorating at least one physical or clinical parameter, which may be indiscernible by the subject. In yet another embodiment, “treating” or “treatment” includes modulating the disease or disorder, either physically or clinically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “patient” refers to a human male or female subject. The methods and uses of the invention described herein are useful to treat a human. In certain embodiments, the term “subject” refers to a “patient.”

As used herein, the term “cell free nucleic acids” refers to the group of nucleic acids, including, but not limited to DNA (cfDNA) and RNA (cfRNA). Cell-free DNA (cfDNA) is DNA circulating freely in bodily fluids such as circulating blood, urine, lymph, interstitial fluid, etc. In some embodiments, cfDNA may be extracted from bodily fluids, such as blood, plasma, and urine. In some embodiments, cfDNA may be arise from apoptotic cells, necrotic cells, and intact cells that are released into the bloodstream or other bodily fluid and eventually lysed. In certain examples the cfDNA is a form of cell-free tumor DNA (ctDNA). In certain embodiments, the cfDNA is referred to synonymously as a cfDNA fragment.

As used herein, the term “single nucleotide variant” or “SNV” refers to a substitution in a single nucleotide at a specific position in the genome.

As used herein, the term “nucleotide insertion” refers to one or more nucleotides inserted into the genome of a biological sample.

As used herein, the term “deletions” refers to one or more nucleotides that has been deleted from genome of a biological sample.

As used herein, the term “translocation” refers to the movement in position of one or more nucleotides to another position in the genome.

As used herein, the term “copy number changes” refers to a sequence of nucleotides that has been duplicated and placed in the genome.

As used herein, the term “primer extension” refers to a technique whereby the 5′ ends of a nucleic acid sequence can be mapped. Primer extension is performed by annealing a specific oligonucleotide primer to a position downstream of a nucleic acid template's 5′ end. Using a reverse transcriptase, the primer can be extended to create a copy of the template.

As used herein, the term “DNA modifications” refers to methylations, hydroxy methylations, and/or strand specific DNA deaminations that may occur to the nucleotides in DNA.

As used herein, a “first target-specific primer” is an oligonucleotide comprising a nucleic acid sequence that can specifically anneal, under suitable annealing conditions, to a target nucleotide sequence of a template nucleic acid. During amplification, the first target-specific primer generates a strand that is complementary to its template, and this complementary strand is capable of being hybridized with a first adapter primer.

As used herein, a “first adapter primer” is an oligonucleotide comprising a nucleic acid sequence that can specifically anneal, under suitable annealing conditions, to a complementary sequence of an adapter nucleic acid. As the first adapter primer is therefore identical to at least a portion of the adapter, it anneals to the complementary strand generated by the first target specific-primer to allow amplification to proceed.

As used herein, a “second target-specific primer” is an oligonucleotide comprising a nucleic acid sequence that can specifically anneal, under suitable annealing conditions, to a portion of the target nucleotide sequence comprised by the amplicon resulting from a preceding amplification step. During amplification, the second target-specific primer generates a strand that is complementary to its template, and this complementary strand is capable of being hybridized with a second adapter primer.

As used herein, a “second adapter primer” is an oligonucleotide comprising a nucleic acid sequence that can specifically anneal, under suitable annealing conditions, to a complementary sequence of an adapter nucleic acid. As the first adapter primer is therefore identical to at least a portion of the adapter, it anneals to the complementary strand generated by the second target specific-primer to allow amplification to proceed.

As used herein, the term “polymerase extension” refers to template-dependent addition of at least one complementary nucleotide, by a nucleic acid polymerase, to the 3′ end of a primer that is annealed to a nucleic acid template.

As used herein, the term “nucleic acid adapter” or “adapter” refers to a nucleic acid molecule that may be ligated to a nucleic acid comprising a target nucleotide sequence to provide one or more elements useful during amplification and/or sequencing of the target nucleotide sequence.

As used herein, the term “nested” is used to describe a positional relationship between the annealing site of a primer of a primer pair and the annealing site of another primer of another primer pair. For example, in some embodiments, a second primer is nested by 1, 2, 3 or more nucleotides relative to a first primer, meaning that it binds to a site on the template strand that is frame-shifted by 1, 2, 3 or more nucleotides.

As used herein, the term “nucleic acid polymerase” refers to an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and are commercially available. One group of nucleic acid polymerases are thermostable, i.e., they retain function after being subjected to temperatures sufficient to denature annealed strands of complementary nucleic acids, e.g., 94° C., or sometimes higher.

As used herein, the term “about” indicates and encompasses an indicated value and a range above and below that value. In certain embodiments, the term “about” indicates the designated value ±10%, ±5%, or ±1%. In certain embodiments, the term “about” indicates the designated value ± one standard deviation of that value.

As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tm).

As used herein, the term “anneal” refers to the formation of one or more complementary base pairs between two nucleic acids.

As used herein, the term “amplification regimen” refers to a process of specifically amplifying (increasing the abundance of) a nucleic acid of interest.

As used herein, “substantially anneal” refers to an extent to which complementary base pairs form between two nucleic acids that, when used in the context of a PCR amplification regimen, is sufficient to produce a detectable level of a specifically amplified product.

In some embodiments, a “buffer” may include solvents (e.g., aqueous solvents) plus appropriate cofactors and reagents which affect pH, ionic strength, etc.

As used herein, “primer” refers to an oligonucleotide capable of specifically annealing to a nucleic acid template and providing a 3′ end that serves as a substrate for a template-dependent polymerase to produce an extension product which is complementary to the template.

As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel.

In this disclosure, methods are presented demonstrating a medical procedure to determine whether DNA has been modified, preferably from a single biological sample.

The present disclosure, also provides a method for diagnosis by determining whether a DNA has been modified by evaluating DNA modification status, preferably with respect to a single biological sample.

The present disclosure, also sets forth processes, in addition to making and using the same, and other solutions to problems in the relevant field.

The present disclosure, also sets forth processes that are advantageous over existing methods to determine the presence of any DNA modifications in a sample. Specifically, the present disclosure sets forth processes that are mutually exclusive with respect to identifying DNA modifications and nucleotide identity from isolated DNA. This thereby preserves the isolated DNA for one or more experiments.

Analyzing DNA for modifications or for sequencing nucleotides often requires chemical manipulation of the DNA. This analysis potentially alters the base pairs in isolated DNA. Therefore, in order to confidently identify a nucleotide change one should manipulate a sample as little as possible to ensure that any observed changes are not a result of a manipulation.

For example, commercially available methods of DNA methylation identification require either: the sequence of a sample be altered via bisulfate conversion, enzymatic deamination, or selectively enriched based on the presence or absence of methylation, or TET-assisted pyridine borane sequencing (TAPS). Bisulfite conversion effectively re-writes all non-methylated cytosines as uracil. The result is the original cytosines being read as thymine during standard DNA sequencing experiments. This potentially introduces confounding factors when analyzing isolated DNA and can obscure mutation identification.

DNA methylation analysis may also be performed using enrichment techniques. Affinity enrichment is a technique that is often used to isolate methylated DNA from the rest of the DNA population. This is usually accomplished by antibody immunoprecipitation methods or with methyl-CpG binding domain (MBD) proteins. One potential drawback of such enrichment techniques is that this technique often ignores contribution of non-methylated sequences and can obscure mutation identification. Therefore, both methods alter the initial material and thereby obscure mutation identification.

The present disclosure sets forth processes that are important in evaluating both the DNA modification status and nucleotide identity on the same fragment. Evaluating both modification status and nucleotide identity on the same cfDNA advantageously increases the specificity of detection and links possible variants to its expression.

Additionally, by evaluating both the modification status and the nucleotide identity on the same cfDNA fragment, quality control preventing experimental error is increased. For instance, if the presence of a specific methylation site is a rare event, and a mutation at the nucleotide position is also a rare event, then observing both events on the same fragment using the methods as set forth, would be unlikely to have occurred by chance. Essentially, observing these two events in-cis reduces the chance that a single observation is likely to result from error and is reflective of the true state.

The present disclosure sets forth processes that are important for conserving scarce samples of DNA isolated from a subject. The conservation is important because it does not have the disadvantage of splitting a DNA sample for separate evaluations and losing vital materials. In general, cfDNA (cell-free DNA) normally is present at relatively low abundance in plasma. For instance, typically 10-50 ng (1500 to 7500 diploid genome equivalents) of cfDNA, from approximately 10 ml of plasma, is recovered from most samples. In conventional techniques, every time a sample is split to perform a new experiment, the sensitivity of the individual assay is sacrificed.

The present disclosure sets forth processes that allow greater efficiency when processing large volumes of samples for genomics studies. For large scale genomic studies, cfDNA isolation requires large volumes of samples and purification reagents. These large volumes present operational challenges to processing large numbers of samples in a timely and cost effective manner. In contrast, the present disclosure allows a user to obtain the same amount, if not more information, from the same or fewer samples.

### Isolating Cell-Free DNA

In some embodiments, the disclosure relates to the preparation of cfDNA for analysis.

In some embodiments, preparative techniques described herein may be useful for analysis of cfDNA. In certain embodiments, cfDNA screening tests can also be used to screen for tumor DNA, for example, as present in the blood of a cancer patient. In certain embodiments, ctDNA is compared to a patient's genome providing minimally-invasive cancer diagnosis, prognosis, and tumor monitoring.

In some embodiments, suitable protocols for the extraction of cfDNA from bodily fluids are used to obtain a cfDNA sample for use in the methods described herein. For example, in some embodiments, a suitable protocol for isolation of cfDNA from blood comprises centrifugation of a blood, serum, or plasma sample, followed by isolation and purification of cfDNA from the sample. In some embodiments, similar steps are performed for analyzing ctDNA, in which blood are processed. In certain embodiments, the blood is processed by centrifugation to remove all the cells, while the supernatant is processed to obtain cfDNA.

In some embodiments, a biological sample comprising methylated DNA is first immunoprecipitated using an antibody to separate methylated DNA from un-methylated DNA.

In some embodiments, techniques described herein are useful for evaluating tumor DNA and mutation detection. In some embodiments, tumor tissue is evaluated to detect cell-free tumor DNA. Cell-free tumor DNA is present in a wide range of cancers but occurs at different levels and mutant allele fractions. For example, ctDNA is highly fragmented to approximately 170 bp. In some embodiments, ctDNA molecules are released by tumor cells and circulate in the blood of cancer patients. In some embodiments, assays using these molecules are used for early tumor detection, monitoring, or detection of resistance mutations.

In some embodiments cell-free fetal DNA (cffDNA) is isolated. In some embodiments, cffDNA originates in trophoblasts, which may be found in the placenta. In some embodiments, non-invasive prenatal testing (NIPT) is used to screen for fetal abnormalities in the X and Y chromosomes and to determine if a woman is at high risk of having a fetus with Down's syndrome (trisomy 21), trisomy 18, or trisomy 13. In some embodiments, the techniques described herein are used for preparing samples of fetal DNA and mutation detection. In some embodiments, the techniques described herein are used on studies focused on detecting paternally inherited sequences to detect fetal DNA. In certain embodiments, primers that have been designed to target the Y chromosome of male fetuses for polymerase chain reaction (PCR) are used.

In some embodiments, differences in gene activation between maternal DNA and fetal DNA are exploited. In some embodiments, epigenetic modifications are made to detect cffDNA. In some embodiments, a hypermethylated promoter is used as a universal fetal marker to confirm the presence of cell-free fetal DNA.

### Amplifying the Sample

In some embodiments, DNA from the biological sample is amplified to produce an amplified DNA sample. In certain embodiments, the DNA is amplified by standard techniques such as PCR.

In some embodiments, the amplified DNA is analyzed for the nucleotide composition. In certain embodiments, the nucleotide composition is analyzed for the presence of SNV (SNVs), nucleotide insertions or deletions (indels), translocations, and copy number changes, or any combination thereof.

In some embodiments, one or more rounds of amplification are used. In certain embodiments, a first round of amplification is conducted using a first target-specific primer and a first adapter primer.

In some embodiments, in the first PCR amplification cycle of the first amplification step, a first target-specific primer is specifically annealed to a template strand of a nucleic acid comprising a target nucleotide sequence. In some embodiments, depending upon the orientation with which the first target-specific primer was designed, a sequence upstream or downstream of the target nucleotide sequence is synthesized as a strand complementary to the template strand.

In some embodiments, if, during the extension phase of PCR, the 5′ end of a template strand terminates in a ligated adapter, the 3′ end of the newly synthesized complementary strand comprises a sequence capable of hybridizing with a first adapter primer. In subsequent PCR amplification cycles, both the first target-specific primer and the first adapter primer are able to specifically anneal to the appropriate strands of the target nucleic acid sequence and the sequence between the known nucleotide target sequence and the adapter are amplified. In some embodiments, a second round of amplification is conducted using a second target-specific primer and a second adapter primer.

In some embodiments, a second target-specific primer is nested relative to a first target-specific primer. In some embodiments, the use of nested adapter primers eliminates the possibility of producing final amplicons that are amplifiable (e.g., during bridge PCR or emulsion PCR) but cannot be sequenced, a situation that can arise during hemi-nested methods. In other situations, hemi-nested approaches using a primer identical to a sequencing primer results in the carry-over of undesired amplification products from the first PCR step to the second PCR step and ultimately yields artificial sequencing reads. In some embodiments, a second target-specific primer is nested with respect to a first target-specific primer by at least 1 nucleotide, e.g., by 1, 2, 3, 4, 5, 6, 7, 8, 9, 10, or more nucleotides. In some embodiments, a second target-specific primer is nested with respect to a first target-specific primer by about 5 nucleotides to about 10 nucleotides, by about 10 nucleotides to about 15 nucleotides, by about 15 nucleotides to about 20 nucleotides, or by about 20 nucleotides or more.

In some embodiments, the techniques described herein comprise the use of one or more nested primers. In some embodiments, the use of nested primers reduces non-specific binding in PCR products due to the amplification of unexpected primer binding sites.

In some embodiments, a second target-specific primer comprises a 3′ portion that specifically anneals to a target nucleotide sequence and a 5′ tail that does not anneal to the target nucleotide sequence. In some embodiments, the 5′ tail comprises a nucleic acid sequence that is identical to a second sequencing primer. In some embodiments, multiple primers (e.g., one or more target specific primers and/or one or more adapter primers) present in a reaction comprises identical 5′ tail sequence portions.

In some embodiments, a 5′ tail comprises a GC-rich sequence. In some embodiments, a 5′ tail sequence comprises at least 50% GC content, at least 55% GC content, at least 60% GC content, at least 65% GC content, at least 70% GC content, at least 75% GC content, at least 80% GC content, or higher GC content. In some embodiments, a 5′ tail sequence comprises at least 60% GC content. In some embodiments, a 5′ tail sequence comprises at least 65% GC content.

In some embodiments, a second round of amplification comprises a second target-specific primer comprising a 5′ tail, a first adapter primer, and an additional primer. In some embodiments, the additional primer comprises a 3′ portion that is identical to the 5′ tail of the second target-specific primer. In some embodiments, the additional primer comprises additional sequences 5′ to the hybridization sequence that comprises barcodes, index adapter sequences, or sequencing primer sites. In some embodiments, the additional primer comprises a generic sequencing adapter/index primer.

In some embodiments, the first and second target-specific primers are substantially complementary to the same strand of the target nucleic acid. In some embodiments, the portions of the first and second target-specific primers that specifically anneal to the known target sequence comprises a total of at least 20 unique bases of the known target nucleotide sequence, e.g., 20 or more unique bases, 25 or more unique bases, 30 or more unique bases, 35 or more unique bases, 40 or more unique bases, or 50 or more unique bases. In some embodiments, the portions of the first and second target-specific primers that specifically anneal to the known target sequence comprises a total of at least 30 unique bases of the known target nucleotide sequence.

In some embodiments, the first adapter primer comprises a nucleic acid sequence identical to about the 20 5′-most bases of the amplification strand of the adapter and the second adapter primer comprises a nucleic acid sequence identical to about 30 bases of the amplification strand of the adapter, with a 5′ base that is at least 1 nucleotide 3′ of the 5′ terminus of the amplification strand.

In some embodiments, an adapter ligated nucleic acid (e.g., a ligation product) is minimal. In such embodiments, a first adapter primer may be used comprises a portion of the adapter nucleic sequence at its 3′ end and then additional sequencer-important information at its 5′ end. In such embodiments, a second adapter primer may be used comprises, at its 3′ end, the 5′ end of the first adapter primer. In such embodiments, the second adapter primer also has a nucleotide sequence that permits sequencing at its 5′ end. In such embodiments, it is possible to produce, using PCR, a library that is sequencer compatible.

### Primers

In some embodiments, primers (e.g., first and second target-specific primers and first and second adapter primers) are designed such that they will specifically anneal to their complementary sequences at an annealing temperature of from about 61 to 72° C., e.g., from about 61 to 69° C., from about 63 to 69° C., from about 63 to 67° C., from about 64 to 66° C. In some embodiments, primers are designed such that they will specifically anneal to their complementary sequences at an annealing temperature of less than 72° C. In some embodiments, primers are designed such that they will specifically anneal to their complementary sequences at an annealing temperature of less than 70° C. In some embodiments, primers are designed such that they will specifically anneal to their complementary sequences at an annealing temperature of less than 68° C. In some embodiments, primers are designed such that they will specifically anneal to their complementary sequences at an annealing temperature of about 65° C. In some embodiments, systems provided herein are configured to alter vessel temperature (e.g., by cycling between different temperature ranges) to facilitate primer annealing.

In some embodiments, the portions of the target-specific primers that specifically anneal to the known target nucleotide sequence will anneal specifically at a temperature of about 61 to 72° C., e.g., from about 61 to 69° C., from about 63 to 69° C., from about 63 to 67° C., from about 64 to 66° C. In some embodiments, the portions of the target-specific primers that specifically anneal to the known target nucleotide sequence will anneal specifically at a temperature of about 65° C. in a PCR buffer.

### Nucleic Acid Extension, Amplification, and PCR

In some embodiments, methods described herein comprise an extension regimen or step. In such embodiments, extension proceeds from one or more hybridized random primers, using the nucleic acid molecules which the primers are hybridized to as templates. Extension steps are described herein. In some embodiments, one or more random primers hybridizes to substantially all of the nucleic acids in a sample, many of which may not comprise a target nucleotide sequence. Accordingly, in some embodiments, extension of random primers occurs due to hybridization with templates that do not comprise a target nucleotide sequence.

In some embodiments, the methods described herein involve a polymerase chain reaction (PCR) amplification regimen, involving one or more amplification cycles. Amplification steps of the methods described herein comprise a PCR amplification regimen, i.e., a set of polymerase chain reaction (PCR) amplification cycles. In some embodiments, exponential amplification occurs when products of a previous polymerase extension serve as templates for successive rounds of extension. In some embodiments, a PCR amplification regimen according to methods disclosed herein may comprise at least one, and in some cases at least 5 or more iterative cycles. In some embodiments, each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available. In some embodiments, methods described herein can comprise linear amplification. For example, in some embodiments, amplification steps performed using nested primers may be performed using linear amplification. In some embodiments, amplification may be conducted using nucleic acid sequence-based amplification (NASBA). For example, in some embodiments, amplification comprises a T7-mediated NASBA reaction.

In some embodiments, a nucleic acid extension reaction involves the use of a nucleic acid polymerase. A non-limiting example of a protocol for amplification involves using a polymerase (e.g., PHOENIX TAQ®, VERASEQ®) under the following conditions: 98° C. for 30 s, followed by 14-22 cycles comprising melting at 98° C. for 10 s, followed by annealing at 68° C. for 30 s, followed by extension at 72° C. for 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations). In some embodiments, slowing the ramp rate (e.g., 1° C./s, 0.5° C./s, 0.28° C./s, 0.1° C./s or slower), for example, from 98° C. to 65° C., improves primer performance and coverage uniformity in highly multiplexed samples. In some embodiments, systems provided herein are configured to alter vessel temperature (e.g., by cycling between different temperature ranges, having controlled ramp up or down rates) to facilitate amplification.

In some embodiments, a nucleic acid polymerase is used under conditions in which the enzyme performs a template-dependent extension. In some embodiments, the nucleic acid polymerase is DNA polymerase I, Taq polymerase, PHOENIX TAQ®, polymerase, PHUSION®, polymerase, T4 polymerase, T7 polymerase, Klenow fragment, Klenow exo-, phi29 polymerase, AMV reverse transcriptase, M-MuLV reverse transcriptase, HIV-1 reverse transcriptase, VERA SEQ ULTRA® polymerase, VERASEQ® HF 2.0 polymerase, EnzScript, or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on an RNA to produce a complementary DNA molecule (RNA-dependent DNA polymerase activity). In some embodiments, a reverse transcriptase is a mouse moloney murine leukemia virus (M-MLV) polymerase, AMV reverse transcriptase, RSV reverse transcriptase, HIV-1 reverse transcriptase, HIV-2 reverse transcriptase, or another appropriate reverse transcriptase.

In some embodiments, a nucleic acid amplification reaction involves cycles including a strand separation step generally involving heating of the reaction mixture. In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), and a carrier (e.g., 0.01 to 0.5% BSA). A non-limiting example of a suitable buffer comprises 50 mM KCl, 10 mM Tris-HCl (pH 8.8 at 25° C.), 0.5 to 3 mM MgCl₂, and 0.1% BSA. A further non-limiting example of a suitable buffer comprises 50 mM KCl, 10 mM Tris-HCl (pH 8.8 at 25° C.), 0.5 to 5 mM (e.g., approximately 0.5 mM, approximately 1 mM, approximately 2 mM, approximately 3 mM, approximately 4 mM, approximately 5 mM) MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid. In some embodiments, annealing involves two complementary or substantially complementary nucleic acid strands hybridizing together. In some embodiments, in the context of an extension reaction, annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tm (e.g., a calculated Tm) of a primer. In some embodiments, an annealing step of an extension regimen involves reducing the temperature following a strand separation step to a temperature based on the Tm (e.g., a calculated Tm) for a primer, for a time sufficient to permit such annealing. In some embodiments, a Tm can be determined using any of a number of algorithms (e.g., OLIGO®, (Molecular Biology Insights Inc. Colorado) primer design software and VENTRO NTI®, (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html)). In some embodiments, the Tm of a primer can be calculated using the following formula, which is used by NetPrimer software and is described in more detail in Frieir, et al. PNAS 1986 83:9373-9377 which is incorporated by reference herein in its entirety.

Tm=ΔH(ΔS+R*ln(C/4))+16.6 log([K+]))=273.15

wherein: ΔH is enthalpy for helix formation; ΔS is entropy for helix formation; R is molar gas constant (1.987 cal/° C.*mol); C is the nucleic acid concentration; and [K+] is salt concentration. For most amplification regimens, the annealing temperature is selected to be about 5° C. below the predicted Tm, although temperatures closer to and above the Tm (e.g., between 1° C. and 5° C. below the predicted Tm or between 1° C. and 5° C. above the predicted Tm) can be used, as can, for example, temperatures more than 5° C. below the predicted Tm (e.g., 6° C. below, 8° C. below, 10° C. below or lower). In some embodiments, the closer an annealing temperature is to the Tm, the more specific is the annealing. In some embodiments, the time used for primer annealing during an extension reaction (e.g., within the context of a PCR amplification regimen) is determined based, at least in part, upon the volume of the reaction (e.g., with larger volumes involving longer times). In some embodiments, the time used for primer annealing during an extension reaction (e.g., within the context of a PCR amplification regimen) is determined based, at least in part, upon primer and template concentrations (e.g., with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds to 2 minutes, or 30 seconds to 2 minutes.

In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed with thermostable polymerases such as Taq polymerase and variants thereof) is performed at 65° C. to 75° C., or 68° C. to 72° C. In some embodiments, methods provided herein involve polymerase extension of primers that are annealed to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions). In some embodiments, polymerases having exonuclease activity (e.g., Taq polymerase) are useful for producing long library fragments.

In some embodiments, primer extension is performed under conditions that permit the extension of annealed oligonucleotide primers. As used herein, the term “conditions that permit the extension of an annealed oligonucleotide such that extension products are generated” refers to the set of conditions (e.g., temperature, salt and co-factor concentrations, pH, and enzyme concentration) under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation.

In some embodiments, a suitable reaction preparation contains one or more salts (e.g., 1 to 100 mM KCl, 0.1 to 10 mM MgCl2), at least one buffering agent (e.g., 1 to 20 mM Tris-HCl), a carrier (e.g., 0.01 to 0.5% BSA), and one or more NTPs (e.g., 10 to 200 μM of each of dATP, dTTP, dCTP, and dGTP). A non-limiting set of conditions is 50 mM KCl, 10 mM Tris-HCl (pH 8.8 at 25° C.), 0.5 to 3 mM MgCl₂, 200 μM each dNTP, and 0.1% BSA at 72° C., under which a polymerase (e.g., Taq polymerase) catalyzes primer extension.

In some embodiments, a suitable reaction preparation contains one or more salts (e.g., 1 to 100 mM KCl, 0.5 to 5 mM MgCl2), at least one buffering agent (e.g., 1 to 20 mM Tris-HCl), a carrier (e.g., 0.01 to 0.5% BSA), and one or more NTPs (e.g, 50 to 350 μM of each of dATP, dTTP, dCTP, and dGTP). A non-limiting set of conditions is 50 mM KCl, 10 mM Tris-HCl (pH 8.8 at 25° C.), 3 mM MgCl₂, 200 μM each dNTP, and 0.1% BSA at 72° C., under which a polymerase (e.g., Taq polymerase) catalyzes primer extension. A further non-limiting set of conditions is 50 mM KCl, 10 mM Tris-HCl (pH 8.8 at 25° C.), 3 mM MgCl₂, 266 μM dATP, 200 μM dCTP, 133 μM dGTP, 200 μM dTTP, and 0.1% BSA at 72° C., under which a polymerase (e.g., Taq polymerase) catalyzes primer extension.

In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, the two, three or four different deoxyribonucleoside triphosphates are present in equimolar, or approximately equimolar, concentrations. In some embodiments, the two, three or four different deoxyribonucleoside triphosphates are present in different concentrations, which have been experimentally determined to be suitable to a particular implementation of the technology.

In some embodiments, nucleic acid amplification involves up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. In some embodiments, a primer is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein (e.g., Invitrogen, Custom DNA Oligos (Life Technologies, Grand Island, N.Y.) or custom DNA Oligos from Integrated DNA Technologies (Coralville, Iowa)).

### Sample Purification

In some embodiments, the isolated cfDNA is purified from other enzymes, primers, or buffer components using any appropriate step or method. In certain embodiments, the isolated cfDNA is purified using a suitable commercially available kit.

In some embodiments, amplification products are isolated from enzymes, primers, or buffer components before any appropriate step of a method. In some embodiments, any suitable method for isolating nucleic acids may be used. In some embodiments, the isolation comprises Solid Phase Reversible Immobilization (SPRI) cleanup. Methods for SPRI cleanup are well known in the art, e.g., Agencourt AMPure XP-PCR Purification (Cat No. A63880, Beckman Coulter; Brea, Calif.). In some embodiments, enzymes are inactivated by heat treatment. In some embodiments, unlabeled dNTPs are removed by enzymatic treatment.

In some embodiments, unhybridized primers are removed from a nucleic acid preparation using appropriate methods (e.g., purification, digestion, etc.). In some embodiments, a nuclease (e.g., exonuclease I) is used to remove primers from a preparation. In some embodiments, such nucleases are heat inactivated subsequent to primer digestion. Once the nucleases are inactivated, a further set of primers may be added together with other appropriate components (e.g., enzymes, buffers) to perform a further amplification reaction.

In some embodiments, steps of the methods provided herein optionally comprise an intervening sample purification step. In some embodiments, a sample purification step comprises a wash step. In some embodiments, a sample purification step comprises SPRI cleanup (e.g., AMPure).

### DNA Shearing/Fragmentation

In certain embodiments, the purified cfDNA is sheared (e.g., mechanically or enzymatically sheared, sheared via nebulizer) to generate fragments of any desired size. In certain non-limiting examples, non-limiting examples of mechanical shearing processes include sonication, nebulization, and AFA® shearing technology available from Covaris (Woburn, Mass.). In certain embodiments, a nucleic acid are mechanically sheared by sonication. In certain embodiments, a target nucleic acid is not sheared or digested. In certain embodiments, nucleic acid products of preparative steps (e.g., extension products, amplification products) are not sheared or enzymatically digested.

### Adapter and Adapter Ligation

In some embodiments, the methods comprise binding the cfDNA with an adapter.

In some embodiments, an adapter is single-stranded. In some embodiments, an adapter is double-stranded. In some embodiments, a double-stranded adapter comprises a first ligatable duplex end and a second unpaired end. In some embodiments, an adapter comprises an amplification strand and a blocking strand. In some embodiments, the amplification strand comprises a 5′ unpaired portion and a 3′ duplex portion. In some embodiments, the amplification strand further comprises a 3′ overhang. In some embodiments, the 3′ overhang is a 3′ T overhang. In some embodiments, the amplification strand comprises nucleotide sequences identical to a first and second adapter primer. In some embodiments, the blocking strand of the adapter comprises a 5′ duplex portion and a non-extendable 3′ portion. In some embodiments, the blocking strand further comprises a 3′ unpaired portion. In some embodiments, the duplex portions of the amplification strand and the blocking strand are substantially complementary and the duplex portion is of sufficient length to remain in duplex form at the ligation temperature.

In some embodiments, the portion of the amplification strand that comprises a nucleotide sequence identical to a first and second adapter primer can be comprised, at least in part, by the 5′ unpaired portion of the amplification strand.

Y-Shaped Adaptors Comprising Unique Barcodes

In some embodiments, the adapter comprises a “Y” shape, i.e., the second unpaired end comprises a 5′ unpaired portion of an amplification strand and a 3′ portion of a blocking strand. The 3′ unpaired portion of the blocking strand is shorter than, longer than, or equal in length to the 5′ unpaired portion of the amplification strand. In some embodiments, the 3′ unpaired portion of the blocking strand is shorter than the 5′ unpaired portion of the amplification strand. Y-shaped adapters have the advantage that the unpaired portion of the blocking strand will not be subject to 3′ extension during a PCR regimen.

Provided herein are compositions that can be used to identify or analyze nucleic acids. For example, in some embodiments, the composition comprises a pool of Y-shaped adaptors, wherein each Y-shaped adaptor comprises a hybridizable portion at one end of the Y-shaped adaptor and a non-hybridizable portion at the opposite end of the Y-shaped adaptor, wherein the hybridizable portion comprises a unique identifiable double-stranded stem barcode of at least two base pairs.

Also provided herein are compositions that comprising a pool of Y-shaped adaptors, wherein each Y-shaped adaptor comprises a hybridizable portion at one end of the Y-shaped adaptor and a non-hybridizable portion at the opposite end of the Y-shaped adaptor, wherein the non-hybridizable portion comprises i) a pre-defined single-stranded barcode of at least two nucleotides, and ii) a random single-stranded barcode of at least two nucleotides on the same strand as the pre-defined single-stranded barcode.

Further provided herein are compositions that can include a pool of Y-shaped adaptors, wherein each Y-shaped adaptor comprises a hybridizable portion at one end of the Y-shaped adaptor and a non-hybridizable portion at the opposite end of the Y-shaped adaptor, wherein the hybridizable portion comprises a unique double-stranded stem barcode of at least two nucleotides, and wherein the non-hybridizable portion comprises i) a pre-defined single-stranded barcode of at least two nucleotides, and ii) a random single-stranded barcode of at least two nucleotides on the same strand as the pre-defined single-stranded barcode.

In some embodiments, the adaptors comprise a pre-defined single-stranded barcode and a random single-stranded barcode on the 5′ strand of the non-hybridizable portion of the adaptor. In certain embodiments, the pre-defined single-stranded barcode and the random single-stranded barcode is on the 3′ strand of the non-hybridizable portion of the adaptor.

In some embodiments, the pre-defined single-stranded barcode is adjacent to the random single-stranded barcode. It is also explicitly contemplated that the pre-defined single-stranded barcode can be separated from the random single-stranded barcode by one or more nucleotides.

In some embodiments, the pre-defined single-stranded barcode comprises naturally occurring bases {e.g., Adenosine (A), Thymidine (T), Guanosine (G), Cytosine (C), and Uracil (U)) or non-naturally occurring bases e.g., aminoallyl-uridine, iso-cytosines, isoguanine, and 2-aminopurine, and be between 1 and about 20 nucleotides long.

In some embodiments, the length of the random barcode comprises between 1 and about 20 nucleotides and it can contain naturally occurring bases {e.g., Adenosine (A), Thymidine (T), Guanosine (G), Cytosine (C), and Uracil (U)), or non-naturally occurring bases e.g., aminoallyl-uridine, iso-cytosines, isoguanine.

In some embodiments, the length of the double-stranded stem barcode comprises between 1 and about 20 nucleotides.

In some embodiments, the double-stranded stem barcode comprises a pre-defined sequence. In certain embodiments, the double-stranded stem barcode comprises random sequence or comprise both a pre-defined sequence and a random sequence.

In some embodiments, the double-stranded barcode comprises natural and non-natural nucleotides, e.g., aminoallyl-uridine, iso-cytosines, isoguanine, and 2-aminopurine, which assists in the detection of the double-stranded barcode.

In some embodiments, each Y-shaped adaptor comprises a primer sequence. The primer sequence is a PCR primer sequence or a sequencing primer sequence. In some embodiments, the primer sequence is on the non-hybridizable portion of the Y-shaped adaptor. In other embodiments, the primer sequence is on the hybridizable portion of the Y-shaped adaptor. In some embodiments, the primer sequence is the same in the entire Y-shaped adaptor pool. In some other embodiments, the primer sequences on one or more Y-shaped adaptors is different from the primer sequences on other Y-shaped adaptors.

In some embodiments, the blocking strand of the adapter comprises a 3′ unpaired portion that is not substantially complementary to the 5′ unpaired portion of the amplification strand, wherein the 3′ unpaired portion of the blocking strand is not substantially complementary to or substantially identical to any of the primers. In some embodiments, the blocking strand comprises a 3′ unpaired portion that does not specifically anneal to the 5′ unpaired portion of the amplification strand at the annealing temperature, wherein the 3′ unpaired portion of the blocking strand will not specifically anneal to any of the primers or the complements thereof at the annealing temperature. In some embodiments, an adapter nucleic acid comprises, at a minimum, a sample index sequence for multiplexing. In certain embodiments, the adapter nucleic comprises a random molecular barcode.

The adaptors disclosed herein and their specific embodiments can be attached to the one or more nucleic acids through the hybridizable (double-stranded) portion of the adaptors. The adaptors can have free or linked single stranded portions. In some embodiments, the method utilizes adaptors with free single stranded portions (Y-shaped adaptors) and covalently linked single-stranded portions (BAL-Seq adaptors) or a combination of two types of adaptors. In some embodiments, the covalently linked single-stranded portions are linked by a linker. The linker may optionally contain a cleavage site, e.g., a restriction enzyme recognition sequence.

In some embodiments, the adaptors have barcodes located according to several distinct embodiments described below. In some embodiments, the adaptors have one or more barcodes on each single-stranded portion and one or more barcodes in the double stranded portion. In some embodiments, each of the barcodes is located (or co-located) in (a) upper single stranded region (containing the 5′-end), (b) lower single stranded region (containing the 3′-end), and (c) the double-stranded region or stem of the Y-shaped adaptor.

### Unique Molecular Identifiers

In some embodiments, the method utilizes unique molecular identifiers (UMIs). In some embodiments, the UMIs are capable of ligating to the ends of the DNA fragment. In some embodiments, the UMI is present adjacent to the sample index position. In some embodiments, the UMI is between and including 3-10 nucleotides. In some embodiments, the UMI is between and including 3-9 nucleotides. In some embodiments, the UMI is between and including 3-8 nucleotides. In some embodiments, the UMI is between and including 3-7 nucleotides. In some embodiments, the UMI is between and including 3-6 nucleotides. In some embodiments, the UMI is between and including 3-5 nucleotides. In some embodiments, the UMI is between and including 3-4 nucleotides.

In some embodiments, the UMIs are on both strands of the adaptor: the upper and the lower strands, or in the double stranded region. In some embodiments, if the UMIs are matched as originating from the same adaptor, double strand sequencing (i.e., pairing single strands is possible) is used. In some embodiments, the UMIs located in the double stranded region are matched by Watson-Crick pairing. In some embodiments, the known-sequence (not random) UMIs present on the single stranded portions are cross-referenced as belonging to the same adaptor molecule.

In some embodiments, the random single-stranded barcode combined with an endogenous barcode provide a unique identifier for each template nucleic acid. In some embodiments, the endogenous barcode comprises a sequence of any length and comprises one or more sets of nucleotide sequences on a nucleic acid. In certain embodiments, the sequences are at different loci of the nucleic acid. In some embodiments, the endogenous barcode comprises a first sequence on an end of the nucleic acid and a second sequence on the opposite end of the nucleic acid. In some embodiments, the endogenous barcode comprises an internal sequence. In certain embodiments, the endogenous barcode comprises a first sequence that is internal, and a second sequence that is on one end of the nucleic acid. In some embodiments, the endogenous barcode comprises a first and a second sequence that are both internal.

In some embodiments, the amplicons derived from the same template nucleic acid contain the same UMIs. These distinct unique identifiers are used to identify and count the distinct template nucleic acids in the original sample. For example, UMIs can be used to count original template nucleic acids containing the same mutations. In some embodiments, UMIs are used to identify and group the amplicons from the same original template nucleic acid.

In some embodiments, the stem barcode is in any portion of the stem of the adaptor. For example, the stem barcode is adjacent to the base pair to which the adaptor attaches on the nucleic acid or one or more base pairs away from the base pair to which the adaptor attaches on the nucleic acid.

In some embodiments, the unique double-stranded stem barcodes identify strands of the nucleic acid. For example, after an adaptor is attached to a nucleic acid, both strands of the resulting nucleic acid contain the unique stem barcode, even though each strand of the nucleic acid may contain different random single-stranded barcodes or different unique identifier. After amplification, the amplicons derived from one strand of the nucleic acid contain the same stem barcode and the same endogenous barcode as the amplicons derived from the other strand of the same nucleic acid. In some embodiments, the stem barcode identifies amplicons derived from the two strands of the same template nucleic acid. In certain embodiments, the unique stem barcodes identify mutations on one strand, but not the other strand, of the nucleic acid. In some other embodiments, mutations that occur on one strand, but not the other strand, of the template nucleic acid are the result of amplification errors and are disregarded as artifact.

In some embodiments, the method comprises using “tandem” sequencing adaptors containing two fundamentally distinct barcodes, which allow tracking of individual DNA molecules to distinguish real somatic mutations arising in vivo from errors introduced during ex vivo procedures including high-throughput sequencing. In some embodiments, adaptors comprise barcodes that include a defined sequence or a random sequence or a combination of a random sequence and a defined sequence. In some embodiments, the single stranded portion of the adaptor includes a barcode consisting of a multiplex sample ID (MID) portion shared among the adaptor molecules in a sample and a barcode unique to each adaptor molecule.

In some embodiments, the unique barcode is a random barcode. Adaptors with such compound barcodes are referred to as “index adaptors.”

In some embodiments, a typical sample multiplexing barcode (MID) is replaced with a degenerate UMI. In another embodiment, a short UMI (2 or more nucleotides) near the ligating end of the adaptor creates an “insert” or internal barcode or internal UMI. By leveraging the distinct genomic coordinates of each molecule, the internal UMIs of the instant invention allow for shorter barcodes, maximizing sequencing throughput. These internal UMIs allow for efficient recovery of duplex molecules, improving by ˜2-fold on similar prior art approaches. The methods presented compare favorably with error suppression methods from the prior art. See to Lou, D. I., et al. High-throughput DNA sequencing errors are reduced by orders of magnitude using circle sequencing. Proc Natl Acad Sci USA 110, 19872-19877 (2013), (“Lou”); Kennedy, S. R. et al. Detecting ultralow- frequency mutations by Duplex Sequencing. Nat Protoc 9, 2586-2606 (2014), (“Kennedy”); and Schmitt, M. W., et al. Detection of ultra-rare mutations by next-generation sequencing. Proc Natl Acad Sci USA 109, 14508-14513 (2012), (“Schmitt”.)

In some embodiments, the cfDNA is labeled with an adapter prior to any amplification.

In some embodiments, the adapter linked cfDNA is separated from amplified DNA. In certain embodiments, this separation can be performed by chromatography or affinity tag techniques. In certain embodiments, the adapter linked biological DNA is subjected to additional analysis of nucleotide composition. In certain embodiments, the adapter linked DNA is analyzed for the presence of SNV, nucleotide insertions or deletions, translocations, or copy number changes, or any combination thereof.

In some embodiments, the method comprises tagging the nucleic acid sample by chemically ligating a tag to the DNA. In some embodiments, this tag is known as a capture moiety. In certain embodiments, the tag comprises: biotin, dual biotin, fluorescently modified bases, alkyne modified base, functional groups allowing orthogonal chemistry, digoxigenin modified base, purification tags, and unique molecular identifiers (UMIs), or any combination thereof.

### End Repair

In some embodiments, nucleic acid end repair is performed on the cfDNA or amplified products. In certain embodiments, the end repair reaction is conducted prior to attaching the adaptors to cfDNA. In certain embodiments, the end repair reaction is conducted after amplification of the adaptor-modified nucleic acids.

In some embodiments, the end repair reaction is conducted prior to fragmenting the DNA. In other embodiments, the end repair reaction is conducted after fragmenting the DNA.

In some embodiments, the end repair reaction is performed by using one or more end repair enzymes. In some embodiments, enzymes for repairing DNA comprise polymerase and exonuclease. For example, a polymerase can fill in the missing bases for a DNA strand from 5′ to 3′ direction. The resulting double-stranded DNA can be the same length as the original longest DNA strand. An Exonuclease can remove the 3′ overhangs. The resulting double-stranded DNA can be the same length as the original shortest DNA strand.

### A-Tailing

In some embodiments, the method comprises performing an A-tailing reaction on the cfDNA to produce a cfDNA with an A-tail. In certain embodiments, the A-tailing reaction is conducted prior to attaching the adaptors to the cfDNA. In some embodiments, the A-tailing reaction is conducted prior to fragmenting the cfDNA. In certain embodiments, the A-tailing reaction is conducted prior to end repair of the cfDNA.

In some embodiments, the A-tailing reaction is performed by using one or more A-tailing enzymes. In certain embodiments, an A residue is added by incubating a DNA fragment with dATP and a non-proofreading DNA polymerase, which adds a single 3′ A residue.

### Immobilizing the Sample

In some embodiments, the ligated material is immobilized on solid matrix. In certain embodiments, the ligated materials are immobilized on a solid matrix by magnetic beads, bio-streptavidin, click chemistry, antibody-dioxigenin, or any combination thereof

In some embodiments, the method comprises producing a copied DNA fragment from the immobilized tagged sample. In certain embodiments, the method comprises evaluating the DNA mutation status of the DNA fragment in the sample from the copied DNA fragment. In certain embodiments, the DNA mutation status provides information on the DNA methylation status.

In certain embodiments, each strand of the DNA fragments in the tagged sample is bound separately to the substrate.

In some embodiments, a binding partner is attached to an insoluble support. Thus, in some embodiments, the molecule of interest may be immobilized on an insoluble support through a selective binding interaction formed between a capture moiety, present on the adapter, and a binding partner of the capture moiety attached to the insoluble support.

In some embodiments, the insoluble support comprises a bead or other solid surface. In some embodiments, the bead is a paramagnetic bead. The use of beads for isolation is well known in the art, and any suitable bead isolation method can be used with the techniques described herein. In some embodiments, beads that are useful for isolation of the molecules of interest are attached to the beads, and the beads are washed to remove solution components not attached to the beads, allowing for purification and isolation. In some embodiments, the beads are separated from other components in the solution based on properties such as size, density, or dielectric, ionic, and magnetic properties.

In some embodiments, the insoluble support is a magnetic bead. Use of beads allows the derivatized nucleic acid capture moiety to be separated from a reaction mixture by centrifugation or filtration, or, in the case of magnetic beads, by application of a magnetic field. In some embodiments, magnetic beads can be introduced, mixed, removed, and released into solution using magnetic fields. In some embodiments, processes utilizing magnetic beads is automated. In some embodiments, the beads are functionalized using well known chemistry to provide a surface having suitable functionalization for attaching a binding partner of a capture moiety. Derivatization of surfaces to allow binding of the capture moiety is conventional in the art. For example, coating of surfaces with streptavidin allows binding of a biotinylated capture moiety. Coating of surfaces with streptavidin has been described in, for example, U.S. Pat. No. 5,374,524 to Miller. In some embodiments, solid surfaces other than beads may be used. In some embodiments, the solid surfaces are planar surfaces, such as those used for hybridization microarrays, or the solid surfaces are the packing of a separation column.

In some embodiments, a binding partner of a capture moiety may be attached to an insoluble support before, simultaneous with, or after binding the capture moiety. In some embodiments, the capture moiety is contacted with a binding partner of the capture moiety while both are in solution. In such embodiments, the capture moiety: binding partner complex is immobilized on an insoluble support by contacting the complex with an appropriately derivatized surface. In some embodiments, the molecule of interest are isolated through a complex formed between a capture moiety attached to the molecule of interest and a binding partner of the capture moiety.

### Analysis of Samples

In some embodiments, the tagged biological DNA remains in its native state until such a time that its sequence composition is altered.

In some embodiments, the DNA is analyzed for chemical modifications. In certain embodiments, the modification is methylation or hydroxymethylation of cytosines at CpG dinucleotides. In certain embodiments, the modification is stand deamination or the presence of N-methyladenine bases.

In some embodiments, the tagged biological DNA is analyzed for methylation. In certain embodiments, the methylation is analyzed by bisulfite conversion. In certain embodiments, the methylation is analyzed by enzymatic deamination. In certain embodiments, the methylation is analyzed by selective enriched based on the presence or absence of methylation. In certain embodiments, the methylation is analyzed by TET-assisted pyridine borane sequencing (TAPS). In certain embodiments, the bisulfite conversion analysis is able to obtain information on the nucleotide composition and modification status on the exact nucleotide. In certain embodiments, methylated DNA residues comprises bisulfite sequencing and enzymatic based methods of DNA based methods of non-methylated Cytosine conversion to Uracil.

In some embodiments, the DNA is processed for identification of methylated or hydroxymethylated C residues by using bisulfite treatment.

In some embodiments, the DNA is processed by enzymatic deamination of unmethylated C residues via APOBEC following treatment of DNA with TET2 enzyme, to produce restriction enzyme cleavage of fragments with unmethylated restriction sites present. In some embodiments, any combination of the previous embodiments may be used.

In some embodiments, the DNA is analyzed for chemical modifications. In certain embodiments, the modification is methylation or hydroxymethylation of cytosines at CpG dinucleotides. In certain embodiments, the modification is strand deamination or the presence of N-methyladenine bases.

In some embodiments the DNA is analyzed for its composition and chemical modifications.

In some embodiments, the method comprises the concurrent analysis of DNA methylation status and the identification of single nucleotide variations.

In some embodiments, the method comprises the concurrent analysis of DNA methylation status and the identification of insertions and/or deletions in cfDNA.

In some embodiments, the evaluation of DNA mutation status identifies single nucleotide variations.

In some embodiments, the method comprises performing primer extension on the sample. In some examples, the primer extension products are subjected to subsequent analysis of DNA modification status. In certain embodiments, the method of any of the previous claims, wherein the bound biological DNA can be processed for analysis of the DNA modification status.

### Sequencing

In some aspects, the technology described herein relates to methods of DNA sequencing. In some embodiments, the sequencing is performed by a next-generation sequencing method. In some embodiments, non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ION Torrent); DNA nanoball sequencing (Complete Genomics); and technologies available from Pacific Biosciences, Intelligen Bio-systems, and Oxford Nanopore Technologies. In some embodiments, the sequencing primers comprise portions compatible with the selected next-generation sequencing method. Next-generation sequencing technologies and the constraints and design parameters of associated sequencing primers are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the sequencing step relies upon the use of a first and second sequencing primer. In some embodiments, the first and second sequencing primers are selected to be compatible with a next-generation sequencing method as described herein.

In some embodiments, methods of aligning sequencing reads to known sequence databases of genomic and/or cDNA sequences are well known in the art, and software is commercially available for this process. In some embodiments, reads (less the sequencing primer and/or adapter nucleotide sequence) which do not map, in their entirety, to wild-type sequence databases are genomic rearrangements or large insertions or deletion mutations. In some embodiments, reads (less the sequencing primer and/or adapter nucleotide sequence) comprising sequences which map to multiple locations in the genome are genomic rearrangements. In some embodiments, a de novo assembly of reads overlapping into contiguous sequences, or “contigs,” are built and utilized in the alignment of sequencing reads. In some embodiments, a hot spot reference is utilized that does not rely on a publicly accessible genomics database.

In some embodiments, genotyping, detection, identification, or quantitation of the ctDNA utilizes sequencing. In some embodiments, sequencing is accomplished using high-throughput systems. In some embodiments, sequencing is performed using the cfDNA described herewithin. In some embodiments, sequence information of the cfDNA sample is obtained by massively parallel sequencing. In some embodiments, massively parallel sequencing is performed on a subset of a genome, e.g., from a subset of cfDNA from the cfDNA sample. In some embodiments, sequence information is obtained by parallel sequencing using flow cells. In some embodiments, primers for amplification are covalently attached to slides in the flow cells and then the flow cells are exposed to reagents for nucleic acids extension and sequencing. In some embodiments, high-throughput sequencing utilizes the technology available from Helicos Biosciences Corp. (Cambridge, Mass.) such as the Single Molecule Sequencing by Synthesis (SMSS) method. In some embodiments, high-throughput sequencing involves the use of technology available by 454 Life Sciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

In some cases, the high-throughput sequencing utilizes next generation sequencing techniques, e.g., using the HiSeq or MiSeq instruments available from Illumina (San Diego, Calif.) This sequencing method is based on the amplification of DNA on a solid surface using fold-back PCR and anchored primers. The sequencing involves a library preparation step. Genomic DNA is fragmented, and sheared ends are repaired and adenylated. Adaptors are added to the 5′ and 3′ ends of the fragments. The fragments are size selected and purified. The sequencing comprises a cluster generation step. DNA fragments are attached to the surface of flow cell channels by hybridizing to a lawn of oligonucleotides attached to the surface of the flow cell channel. The fragments are extended and clonally amplified through bridge amplification to generate unique clusters. The fragments become double stranded, and the double stranded molecules can be denatured. Multiple cycles of the solid-phase amplification followed by denaturation creates several million clusters of approximately 1,000 copies of single-stranded DNA molecules of the same template in each channel of the flow cell. Reverse strands are cleaved and washed away. Ends are blocked, and primers hybridize to DNA templates. Hundreds of millions of clusters are sequenced simultaneously. Primers, DNA polymerase, and four fluorophore-labeled, reversible terminator nucleotides are used to perform sequential sequencing. All four bases compete with each other for the template. After nucleotide incorporation, a laser is used to excite the fluorophores, and an image is captured and the identity of the first base is recorded. The 3′ terminators and fluorophores from each incorporated base are removed and the incorporation, detection and identification steps are repeated. A single base can be read each cycle. In some embodiments, a HiSeq system {e.g., HiSeq 2500, HiSeq 1500, HiSeq 2000, or HiSeq 1000) is used for sequencing.

In some embodiments, high-throughput sequencing of cfDNA takes place using AnyDot-chips (Genovoxx, Germany), which allows monitoring of biological processes {e.g., miRNA expression or allele variability (SNP detection)). For example, the AnyDot-chips allow for 10×-50× enhancement of nucleotide fluorescence signal detection. Other high- throughput sequencing systems include those disclosed in Venter, J., et al. Science 16 Feb. 2001; Adams, M. et al, Science 24 Mar. 2000; and M. J, Levene, et al. Science 299:682-686, January 2003; as well as U.S. Application Pub. No. 2003/0044781 and 2006/0078937. The growing of the nucleic acid strand and identifying the added nucleotide analog may be repeated so that the nucleic acid strand is further extended and the sequence of the target nucleic acid is determined.

In some embodiments, the methods disclosed herein comprise conducting a sequencing reaction based on one or more genomic regions from a selector.

In some embodiments, the sequencing information is obtained for a subset of genomic regions from a selector. For example, sequencing information may be obtained for 10-500 or more genomic regions from a selector.

In some embodiments, sequencing information is obtained for less than 5%, or up to 95% of the genomic regions from a selector.

### Diseases

In some embodiments, the diseases that may be analyzed by the methods herein, include, but are not limited to, those disease that have changes in the DNA methylation of parts or all of a genome.

In some embodiments, the diseases that may be analyzed by this method include, but are not limited to, cancer. In certain embodiments, the cancers that may be analyzed include, but are not limited to, ovarian, breast, gastric, colon, endometrial, lung, head, neck, colorectal, esophageal, prostate, uterine, pancreatic, kidney, and lymphomas.

In some embodiments, the diseases that may be analyzed include but are not limited to, various neurological conditions. In certain embodiments, the neurological conditions that may be analyzed include, but are not limited to Parkinson's disease, Alzheimer's disease, and Huntington's disease.

### Methods of Treatment Diseases

In some embodiments, the methods described herein to treat various diseases provide data on methylation patterns to a medical professional, who in turn decides on a specific course of treatment.

In some embodiments the treatment may include, but is not limited to, antibody therapies, small molecule therapies, radiation therapies, and cellular therapies. The nature of treatment will be understood to those of skill in the art, especially as treatments and therapies advance.

In some embodiments, the methods described herein may be used by a medical professional to treat various diseases by combining any of the previous embodiments with known treatments and methods of chemical modification analysis.

In some embodiments, the methods described herein may be used by a medical professional for cancer recurrence testing, treatment response monitoring, and asymptomatic early detection, or any combination thereof.

In some embodiments, the methods described herein may be used by a medical professional to decide to not to administer a treatment. In certain embodiments, the methods herein may be used by a medical professional to decide to stop administering a treatment.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

The patent or application file contains at least one drawing executed in color. Copies of this patent or patent application publication with color drawing(s) will be provided by the Office upon request and payment of the necessary fee.

### EXAMPLES

### Method 1: Exemplary Protocol for Methylation Analysis

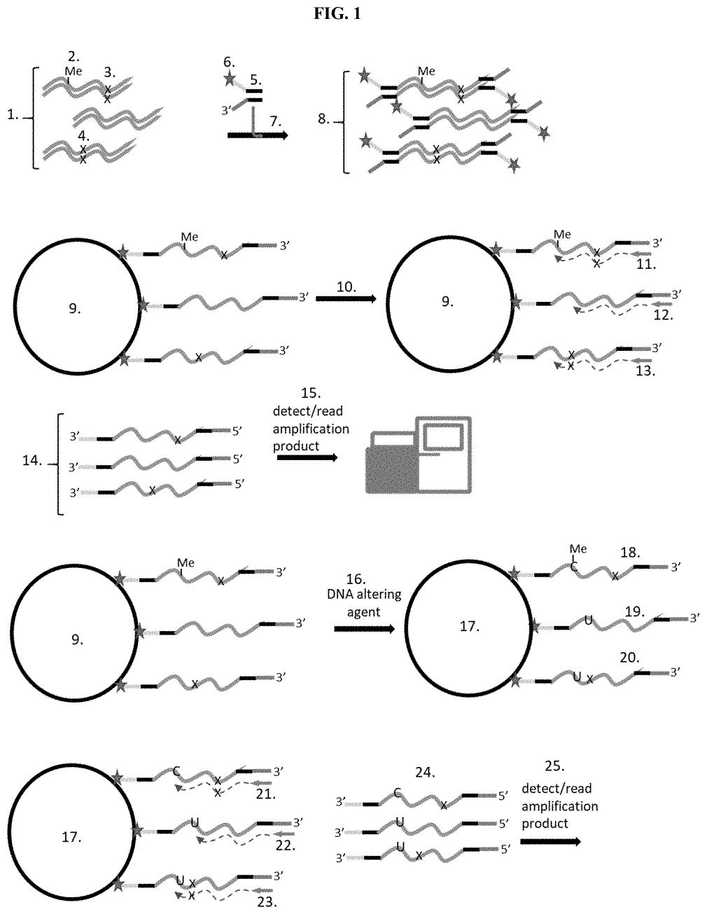

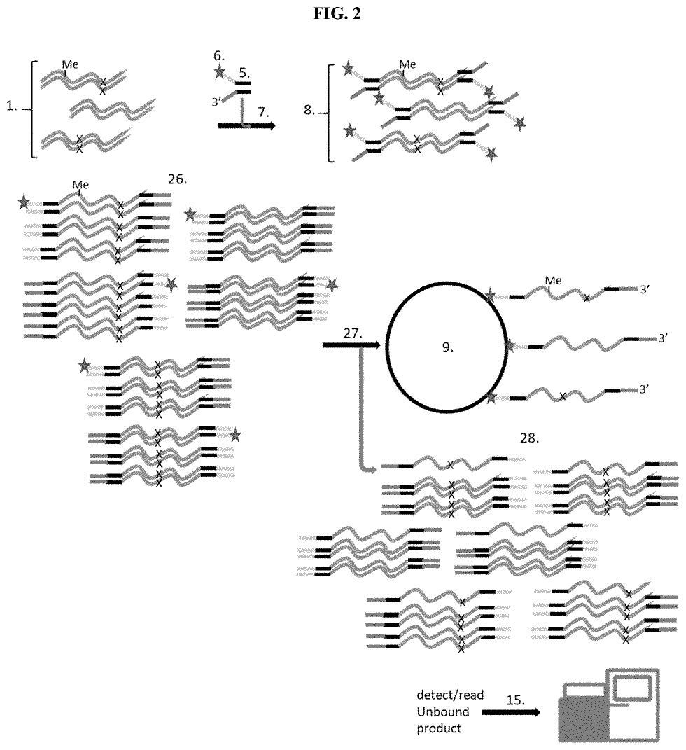

FIGS. 1-2 depict exemplary protocols. Each Figure number represents a successive step in the exemplary protocols and are described in the Figure legends.

The feasibility of the methods was demonstrated by showing that by labelling biological DNA with an affinity reagent, in this case a biotinylated adapter DNA molecule, that methods amplified the labeled DNA producing an enzymatic DNA product. The product of this reaction is primarily made up of the amplified material; however, all of the labeled biological DNA is also present.

The labeled biological DNA was captured using an affinity matrix, in this case a streptavidin coated magnetic bead. Amplified enzymatic DNA is not bound to the affinity matrix and its sequence composition can be determined by currently available methods or technologies. Bead bound DNA, which apart from minute amounts of non-specifically adhered material, is otherwise entirely comprised of the original biological DNA. This DNA carried epigenetic methylation marks, the locations of which were determined by subjecting the bead bound DNA to enzymatic conversion of unmethylated Cytosine to Uracil. Since methylated Cytosines are protected from this conversion, the methylation status was determined by reading Cytosine residues in DNA as such in subsequent analyses.

### Method 2: Molecular Analysis

In order to demonstrate proof of concept for our methods, the methylation status of a region of the BRCA1 promoter was measured in two cell lines, SW620 and OVCAR8. The SW620 cell line lack detectable methylation in a specific region (Clovis Oncology—Personal communication), while in the OVCAR8 cell line, 2 of the same 3 BRCA1 loci are methylated (Kondrashova et al, 2018). The methylation status of the BRCA1 promoter region was assessed in these samples following enzymatic conversion of non-methylated Cytosines to Uracil using hydrolysis probe-based allele specific PCR (Swisher et al, 2021). Approximately 66% methylated BRCA1 promoter in OVCAR8 cells and 0% methylated BRCA1 promoter in SW620 was expected to be observed in cells if conversion is efficient.

DNA from the two cell lines was fragmented by sonication using a Covaris LE220 instrument to an average size of 240 bp. Following fragmentation, 50 ng of DNA from these cell lines was end-repaired and dA tailed using conventional methods, i.e. T4 DNA polymerase and T4 polynucleotide kinase for end-repair and Taq DNA polymerase for addition of the single dA base 3′ overhang (Agilent Technologies). End-repaired and dA tailed DNA was then ligated to a custom double stranded DNA adapter (Integrated DNA Technologies). This adapter was synthesized such that every Cytosine residue in the adapter was methylated and the 5′ end of the adapter strand distal to the ligation site carried a 5′ biotin modification. Following the short ligation reaction incubation, the ligated product was purified using SPRI beads (Beckman Coulter) to remove free unligated adapter and the other non-DNA reaction components. Purified ligation product was amplified with 6 cycles of PCR using primers that added sample identification indices and sequencing adapters for an Illumina sequencing machine (Agilent Technologies). The original biological DNA was recovered from the product of the PCR by binding to Streptavidin coated magnetic beads (ThermoFisher Scientific).

The unbound material was separated from the bead bound material, purified using SPRI beads, and quantified using Quant-it reagent dsDNA assay kit (ThermoFisher Scientific). The unbound material represents amplification product of the biological DNA and carries no epigenetic marks. Bead bound DNA was washed with Hybridization capture wash solutions according to manufacturer recommendations (Integrated DNA Technologies) then subjected to enzymatic Cytosine conversion using a slightly modified workflow of the NEBNext Enzymatic Methyl-seq Kit (New England Biolabs). Following the deamination step of the workflow, converted bead bound DNA was washed with 80% Ethanol twice on beads. Converted bead bound DNA was amplified with a 6 cycle PCR using primers that added sample identification indices and sequencing adapters for an Illumina machine and a Uracil tolerant polymerase, i.e. Q5U (New England Biolabs). ing of converted DNA was used in allele specific PCR to assess methylation status of the BRCA1 promoter (Swisher et al, 2021). Non-bead bound DNA was used as a control to assess the efficacy of performing the conversion on beads. The yield of the first amplification product was compared to the second amplification product to assess our recovery.

### Results: Statistical Analysis

Table 1 shows the estimated percent recovery of biological DNA following capture on Streptavidin beads, enzymatic conversion of unmethylated Cytosines to Uracils, and amplification by PCR.

TABLE 1

| | Library PCR | Post Conversion | |
|---|---|---|---|
| | (KAPA + | PCR (NEB Q5U + | Approx. % |
| Sample ID | IDT UDIs) | IDT UDIs) | Recovery |
| OVCAR8_bead_rep1 | 14.10 | 5.13 | 36% |
| OVCAR8_bead_rep2 | 15.60 | 7.36 | 47% |
| OVCAR8_bead_rep3 | 28.80 | 10.10 | 35% |
| OVCAR8_bead_rep4 | 28.40 | 10.10 | 36% |
| SW620_bead_rep1 | 11.30 | 10.50 | 93% |
| SW620_bead_rep2 | 10.30 | 7.14 | 69% |
| SW620_bead_rep3 | 14.90 | 8.30 | 56% |
| SW620_bead_rep4 | 13.40 | 7.79 | 58% |

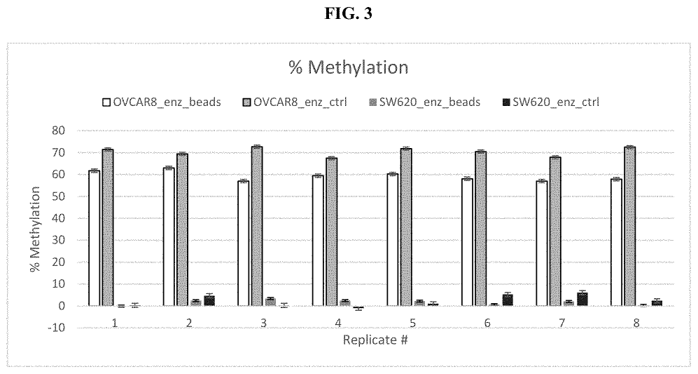

A low % of methylation was observed at the BRCA1 promoter in SW620 DNA that has undergone unmethylated Cytosine conversion to Uracil (FIG. 3, % methylation SW620_enz_beads) indicates that this process does occur on material that has been captured on Streptavidin beads. Additionally, the observation that we measure OVCAR8 BRCA1 promoter as being approximately 66% methylated (FIG. 3, OVCAR8_enz_beads) indicates that the material that was captured on the Streptavidin beads contains methylated Cytosine residues at the predicted levels. Therefore, the evidence suggests that captured DNA contains methylated residues and is therefore very likely comprised of the original biological DNA—thereby supporting the efficacy of our methods.

The efficiency of the recovery of methylated DNA, i.e. the biological DNA, was estimated by quantifying the yield of library PCR prior to and subsequent to capture on Streptavidin coated magnetic beads and enzymatic conversion of non-methylated Cytosines (Table 1). The estimation of recovery is likely to be conservative in that the amplification of converted DNA was performed in the presence of Streptavidin coated magnetic beads which if anything can have inhibitory impacts on PCR. Regardless, we observed greater than 33% recovery of original biological DNA with this simple assessment.

Together, these data demonstrate that the methods described herein are a viable means of obtaining information on both the methylation status and mutation status from the same sample of nucleic acid.

### Example 2: Patient and Case Selection

In one example, a medical professional obtains isolated ctDNA from a subject, and uses the methods described in any of previous embodiments to detect the relative abundance of ctDNA and any chemical modifications to the DNA. The medical professional administers a treatment to the subject and monitors the relative abundance of the ctDNA and any chemical modifications, mutations, or any combination thereof to determine the success of a treatment regimen.

In one example, if a treatment is successful, then ctDNA concentrations are observed to decline from baseline levels.

### REFERENCES

The references cited in this disclosure are incorporated by their entirety.

Alborelli I. et al. Cell-free DNA analysis in healthy individuals by next-generation sequencing: a proof of concept and technical validation study. Cell Death and Disease. 2019. 10:534.

Newman A. M. et al. Integrated digital error suppression for improved detection of circulating tumor DNA. Nature Biotechnology. 2016. 34(5): 547-555.

Schmitt et al. Detection of ultra-rate mutations by next-generation sequencing. PNAS. 2012. 109: 14508-14513.

Stahl et al. Methods of nucleic acid sample preparation for analysis of cell-free DNA. U.S. Pat. No. 10,683,531 B2.

Diehn et al. Identification of circulating nucleic acids. WIPO WO 2016/040901.

Vogelstein et al. Safe sequencing system. U.S. Pat. No. 9,476,095 B2.

Liu Y. et al. Bisulfite-free direct detection of 5-methylcytosine and 5-hydroxymethylcytosine at base resolution. Nature Biotechnology. 2019. 37:424-429.

Chiange, Jiang Wang et al. BRCA1 promoter methylation predicts adverse ovarian cancer prognosis. Gynecologic Oncology. 2006. 101(3) 403-410.

Kondrashova, O. et al. Methylation of all BRCA1 copies predicts response to the PARP inhibitor rucaparib in ovarian carcinoma. Nature Communications. 2018. 9: 3970.

Esteller, M. CpG island hypermethylation and tumor suppressor genes: a booming present, a brighter future. Oncogene. 2002. 21: 5427-5440.

Butler, T. M. et al. Circulating tumor DNA dynamics using patient-customized assays are associated with outcome in neoadjuvantly treated breast cancer. Molecular Case Studies. 2019. Abstract.

Adar, T. et al. A tailored approach to BRAF and MLH1 methylation testing in a universal screening program for Lynch syndrome. Modern Pathology. 2017. 30: 440-447.

Liu M. C. et al. Sensitive and specific multi-cancer detection and localization using methylation signatures in cell-free DNA. Annals of Oncology. 2020. 31(6): 745-759.

Ritch, E. et al. Predicting therapy response and resistance in metastatic prostate cancer with circulating tumor DNA. Urologic Oncology: Seminars and Original Investigations. 2018. 36(8): 380-384.

Boonstra, P. A. et al. Clinical utility of circulating tumor DNA as a response and follow-up marker in cancer therapy. Cancer and Metastasis Reviews. 2020. 39, 999-1013.

Kondrashova, Olga et al. “Methylation of all BRCA1 copies predicts response to the PARP inhibitor rucaparib in ovarian carcinoma.” Nature communications vol. 9,1 3970. 28 Sep. 2018, doi:10.1038/s41467-018-05564-z

Swisher, Elizabeth M et al. “Molecular and clinical determinants of response and resistance to rucaparib for recurrent ovarian cancer treatment in ARIEL2 (Parts 1 and 2).” Nature communications vol. 12,1 2487. 3 May. 2021, doi:10.1038/s41467-021-22582-6

Gordevic̆ius, J., Narmonte, M., Gibas, P. et al. Identification of fetal unmodified and 5-hydroxymethylated CG sites in maternal cell-free DNA for non-invasive prenatal testing. Clin Epigenet 12, 153 (2020). doi.org/10.1186/s13148-020-00938-x

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A method for assessing DNA modification status of a nucleic acid sample from a subject with respect to a single sample from a subject suspected of having a disease, the method comprising:
(a) obtaining a nucleic acid sample from the subject;
(b) tagging a DNA fragment of the nucleic acid sample from the subject to create a tagged DNA fragment;
(c) immobilizing the tagged DNA fragment to a substrate, wherein each strand of the tagged DNA fragment in the nucleic acid sample is bound separately to the substrate;
(d) evaluating DNA mutation status of the tagged DNA fragment;
(e) evaluating DNA modification status of the tagged DNA fragment; and
(f) assessing the DNA modification status of the nucleic acid sample.

14. A method for diagnosing a subject with a potential disease by evaluating DNA modification status of a nucleic acid sample from a subject with respect to a single sample suspected of having a disease, the method comprising:
(a) obtaining a nucleic acid sample from a subject;
(b) tagging a DNA fragment of the nucleic acid sample from the subject to create a tagged DNA fragment;
(c) immobilizing the tagged DNA fragment to a substrate, wherein each strand of the tagged DNA fragment in the nucleic acid sample is bound separately to the substrate;
(d) evaluating DNA mutation status of the tagged DNA fragment;
(e) evaluating DNA modification status of the tagged DNA fragment; and
(f) diagnosing a subject with a potential disease by evaluating the DNA modification status of the nucleic acid sample.

15. The method of claim 13, wherein the nucleic acid sample comprises cfDNA.

16. The method of claim 13, wherein the DNA modification status of the DNA fragment in the nucleic acid sample is evaluated for the presence of methylations, hydroxy methylations, strand specific deaminations, or N-methyladenine bases on the DNA fragment.

17. The method of claim 13, wherein the DNA mutation status of the DNA fragment in the nucleic acid sample is evaluated based on single nucleotide variations, insertions, translocations, copy number, or deletions present in the DNA fragment.

18. The method of claim 13 further comprising producing a copied DNA fragment from the immobilized tagged sample, wherein the copied DNA fragment is produced by:
(a) immobilizing the DNA fragment to a substrate to create an immobilized DNA fragment;
(b) copying the DNA fragment through PCR amplification to create a copied DNA fragment; and
(c) separating the copied DNA fragment from the immobilized DNA fragment.

19. The method of claim 13, wherein each strand of the DNA fragment is immobilized separately to the substrate.

20. The method of claim 13 further comprising evaluating the DNA mutation status of the DNA fragment in the sample from the copied DNA fragment.

21. The method of claim 13, wherein each strand of the DNA fragment in the tagged sample is bound separately to the substrate.

22. The method of claim 13, wherein the DNA mutation status provides information on the DNA methylation status.

23. The method of claim 13, wherein the evaluation of DNA mutation status identifies single nucleotide variations.

24. The method of claim 13, wherein the evaluation of DNA mutation status identifies insertions or deletions present in the cfDNA.

25. The method of claim 13, further comprising treating the subject for their potential condition or disease.

26. The method of claim 13 wherein tagging the nucleic acid sample is accomplished by chemically ligating a tag to the DNA.

27. The method of claim 26, wherein the tag comprises: biotin, dual biotin, fluorescently modified bases, alkyne modified base, functional groups allowing orthogonal chemistry, digoxigenin modified base, purification tags, and unique molecular identifiers (UMIs).

28. (canceled)

29. (canceled)

30. (canceled)

31. (canceled)

32. The method of claim 13, wherein the ligated material is immobilized on solid matrix by magnetic beads, bio-streptavidin, click chemistry, or antibody-dioxigenin.

33. The method of claim 13, wherein the copied DNA fragment can be subjected to subsequent analysis of DNA modification status.

34. The method of claim 13, wherein the immobilized DNA fragment can be processed for analysis of the DNA modification status.

35. (canceled)

36. (canceled)

37. (canceled)

38. (canceled)

39. The method of claim 34, wherein the DNA is treated with sodium bisulfite.

40. The method of claim 13, wherein the methods for detection of methylated DNA residues comprises bisulfite sequencing and enzymatic based methods of DNA based methods of non-methylated Cytosine conversion to Uracil.

41. (canceled)

42. (canceled)

43. (canceled)

44. The method of claim 13, further comprising:
(a) copying the tagged cfDNA molecule through PCR amplification to produce an untagged copy;
(b) binding the tagged cfDNA molecule to the substrate;
(c) separating the tagged cfDNA from the untagged copy; and
(d) analyzing the mutation status of the unbound material.
